(19) United States
(12) Patent Application Publication
(10) Pub. No.: US 2011/0295005 A1
(43) Pub. Date: Dec. 1, 2011

(54) PROCESS FOR PREPARING PYRIMIDINE DERIVATIVES

(75) Inventors: Armin Boerner, Rostock (DE); Gerd Koenig, Zwickau (DE); Natalia Andrushko, Rostock (DE); Vasyl Andrushko, Rostock (DE)

(73) Assignee: Ratiopharm GmbH, Ulm (DE)

(21) Appl. No.: 12/674,253

(22) PCT Filed: Aug. 19, 2008
(86) PCT No.: PCT/EP08/06806

§ 371 Date: Feb. 19, 2010

(30) Foreign Application Priority Data
Aug. 20, 2007 (EP) 07016279.7

(51) Int. Cl. C07D 239/42

(52) U.S. Cl. 544/297

(57) ABSTRACT

The present invention relates to a process for preparing pyrimidine derivatives of the formula V, in particular as intermediates useful for preparing pyrimidine derivatives of a class that is effective at inhibiting the biosynthesis of cholesterol in humans, such as HMG-CoA reductase inhibitors, e.g. rosuvastatin.

The present invention relates to a process for preparing pyrimidine derivatives as intermediates useful for preparing pyrimidine derivatives of a class that is effective at inhibiting the biosynthesis of cholesterol in humans, and more particularly to improved synthetic methods for preparing rosuvastatin.

It is known that certain 3,5-dihydroxy heptanoic acid derivatives are competitive inhibitors of the 3-hydroxy-3-methyl-glutaryl-coenzyme A (“HMG-CoA”). HMG-CoA is a key enzyme in the biosynthesis of cholesterol in humans. Its inhibition leads to a reduction in the rate of biosynthesis of cholesterol. The first HMG-CoA inhibitor to be described is compactin ([1S-[1α(R*), 7β,8β(2S*,4S*),8αβ]]-1,2,3,7,8a-hexahydro-7-methyl-8-[2-(tetrahydro-4-hydroxy-6-oxo-2H-pyran-2-yl)pethyl]-1-naphthalenyl 2-methylbutanoate), which was isolated from cultures of Penicillium in 1976. In 1987, lovastatin ([1S-[1α(R*),3α,7β,8β(2S*,4S*),8αβ]]-1,2,3,7,8,8a-hexahydro-3,7-dimethyl-8-[2-(tetrahydro-4-hydroxy-6-oxo-2H-pyran-2-yl)pethyl]-1-naphthalenyl 2-methylbutanoiate) became the first HMG-CoA reductase inhibitor approved by the Food and Drug Administration (FDA) for treatment of hypercholesterolemia. Both compactin and lovastatin are derived from bacterial cultures. Two other naturally-derived HMG-CoA reductase inhibitors, simvastatin and pravastatin are structurally related to compactin and lovastatin.

Another known HMG-CoA reductase inhibitor which can be used for the treatment of, inter alia, hypercholesterolemia and mixed dyslipidemia is rosuvastatin. Rosuvastatin has the chemical name (E)-7-[4-(4-fluorophenyl)-6-isopropyl-2-[methyl(methylsulfonyl)amino]pyrimidin-5-yl](3R,5S)-3,5-dihydroxyhept-6-enoic acid and the structural formula

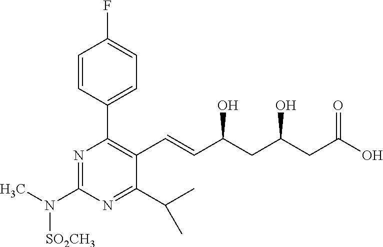

Rosuvastatin calcium is marketed under the trademark CRESTOR™.

In contrast to compactin, lovastatin, simvastatin and pravastatin, there is no known fermentation culture that produces rosuvastatin. It must therefore be synthesized by traditional synthetic methods.

A number of processes for the synthesis of rosuvastatin and derivatives thereof are known. Some of the processes are concerned with the synthesis of the 3,5-dihydroxy hepten-6-oic acid side chain of the pyrimidine ring while others are concerned with the formation of the pyrimidine ring or the linkage of the side chain to the pyrimidine ring.

In the synthesis of rosuvastatin for the formation of the double bond in the C7 side chain, the application of the Wittig reaction has long been found to be advantageous (cf. Scheme 1).

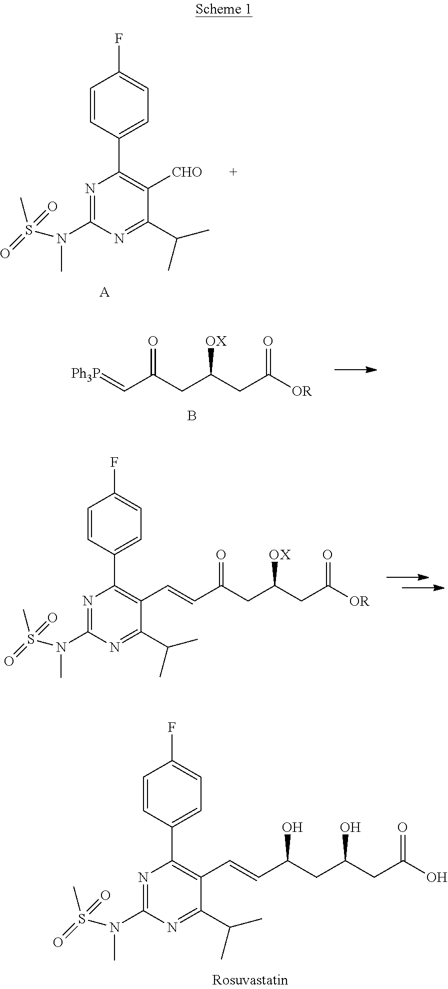

U.S. Pat. No. 5,260,440 discloses the reaction of methyl(3R)-3-(tert-butyldimethylsilyloxy)-5-oxy-6-triphenylphosphoranyliden hexanoic acid derivatives (cf. compound B of Scheme 1, X=t-butyldimethylsiloxy) with 4-(4-fluorophenyl)-6-isopropyl-2-(N-methyl-N-methylsulfonylamino)-5-pyrimidine-carboxaldehyde (cf. compound A of Scheme 1), followed by a deprotection step, a reduction step and a hydrolysis step to obtain rosuvastatin.

WO 00/49014 discloses the synthesis of rosuvastatin via a Wittig reaction using a Wittig reagent which comprises the pyrimidine core of the rosuvastatin molecule. However, the preparation of such Wittig reagents is disadvantageous, in particular as in the reaction steps to obtain the Wittig reagent the expensive fully substituted pyrimidine compound has to be used, and low yields therefore means high costs of the synthesis.

WO 03/097614 also discloses the synthesis of rosuvastatin via a Wittig reaction. The aldehyde corresponding to compound A above is synthesized following reduction and oxidation steps according to scheme 2 depicted below.

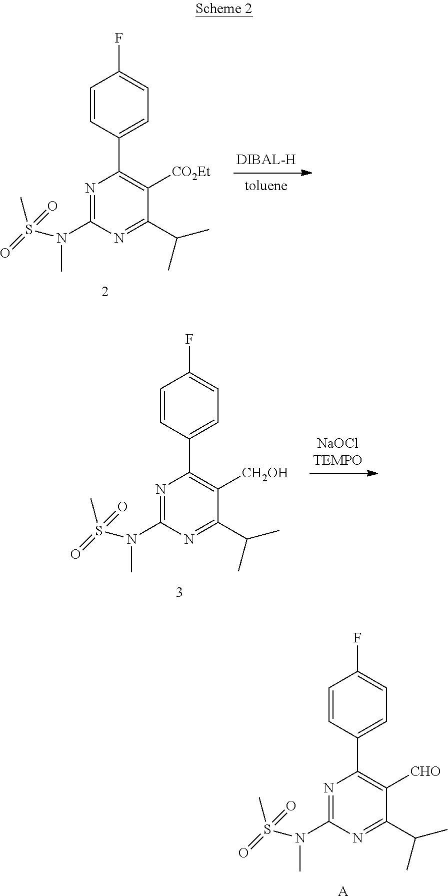

This approach disclosed in WO 03/097614 or similar known approaches to obtain aldehyde A by reduction and/or oxidation processes from compound (2) and/or (3) or derivatives thereof is disadvantageous, as many reduction steps are involved, which often have low yield and much of the expensive fully substituted pyrimidine compound is lost, e.g. due to the formation of by-products.

Therefore there is still a need for further methods of synthesizing pyrimidine intermediates and in particular pyrimidine intermediates for the preparation of rosuvastatin.

It has now been found that several prior art problems can surprisingly be overcome by a certain process for the preparation of pyrimidine intermediates, in particular such as compound A, which can then be subjected to a Wittig reaction, in particular for the synthesis of rosuvastatin. In particular it has been found that said pyrimidine intermediates can for example be synthesized according to the following reaction scheme 3.

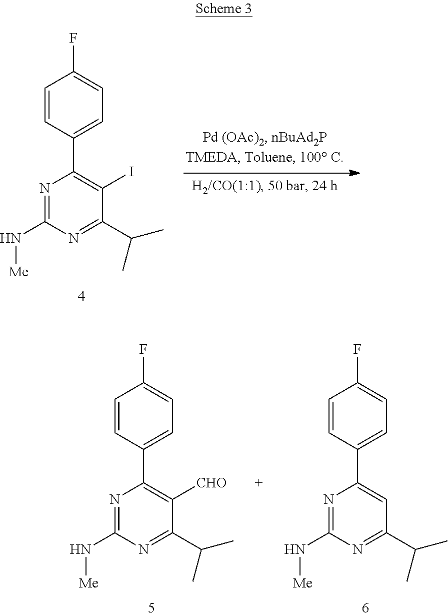

In particular it was surprisingly found that the 5-formyl-pyrimidine derivative (5) can be easily obtained by a formylation of the corresponding 5-iodo-pyrimidine compound in excellent yields.

This sequence has the advantage that the undesirable multiple oxidation and reduction steps according to scheme 2 can be avoided in the synthesis of the 5-formyl-pyrimidine derivatives (e.g. 5 or compound A).

Therefore, the present invention relates to a process for the preparation of a compound of the formula V

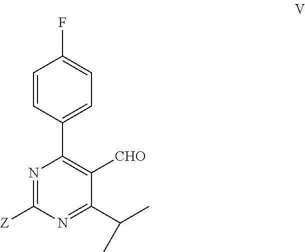

wherein Z is a —NMeSO₂Me group or a group capable of being converted into a —NMeSO₂Me group,
which process comprises the steps of
   a) formylation of a compound of the formula VIII

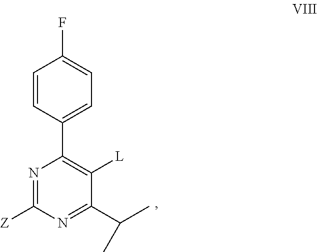

wherein Z is defined as above and L is a leaving group and
   b) optionally converting Z into a —NMeSO₂Me group.

The compound of formula V prepared by the process of the present invention is intended as intermediate for the preparation of pyrimidine derivatives having HMG-CoA reductase inhibition activity as described above, in particular rosuvastatin.

Residue Z is a —NMeSO₂Me group or a group capable of being converted into a —NMeSO₂Me group. The term —NMeSO₂Me group means a residue as depicted in the following formula X

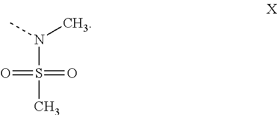

Groups capable of being converted into a —NMeSO₂Me group means that the group is selected from any functional group which can be converted, by carrying out one or more chemical steps, to form a —NMeSO₂Me group. Suitable groups which are capable of being converted, and the chemical synthesis steps that can be used to carry out the conversion are well known in the art, and are e.g. described in WO 2006/067456, the disclosure of which is incorporated herein by reference. Preferred groups capable of being converted into a —NMeSO₂Me group are hydroxy, C₁₋₁₀ alkoxy, halogen (in particular chloro), tosyloxy, amino, C₁₋₁₀ alkylamino, such as methylamino, C₁₋₁₀ dialkylamino and methyl sulfonylamino groups.

Residue L is a leaving group, and in particular a leaving group suitable for a formylation reaction wherein the leaving group, which is bound to the pyrimidine heterocycle, is replaced by a formyl group. Suitable leaving groups are known in the art and are e.g. halogen, such as chlorine, bromine or iodine, the latter being particularly preferred, but also tosyl (toluol sulfonyl), mesyl (methyl sulfonyl) or further known leaving groups. Regarding further known leaving groups it is referred to the German patent application No. DE 10 2005 022284.6 A1, the disclosure of which is incorporated herein by reference.

In a preferred embodiment of the present invention the formylation step is carried out in the presence of a catalyst, in particular in the presence of a metal or transition metal catalyst, most preferred a palladium based catalyst. Preferably the formylation is carried out in the presence of palladium based catalysts. In particular the formylation catalyst is prepared in situ by reacting a suitable soluble palladium compound with a suitable ligand, in particular a phosphine ligand, e.g. the formylation catalyst is a catalyst prepared in situ from Pd(OAc)₂ and nBuAd₂P. “nBu” means n-butyl and “Ad” means adamantyl. Other suitable catalysts are known in the art. For the Pd-catalyzed formylation of aryl-bromides, see e.g. ref.: [S. Klaus, H. Neumann, A. Zapf, D. Strübing, S. Hübner, J. Almena, T. Riermeier, P. Groβ, M. Sarich, W.-R. Krahnert, K. Rossen, M. Beller, Angew. Chem. Int. Ed. 2006, 45, 154-158.]

The formylation reaction is typically conducted using hydrogen gas (H₂) and carbon monoxide gas (CO) in suitable molar ratio, e.g. about 5:1 to about 1:5, more preferred about 2:1 to about 1:2, in particular in a ratio of about 1:1. The formylation reaction is conducted at usual temperatures known to the person skilled in the art, preferably at increased temperatures of about 80 to about 120° C., in particular at about 100° C. Preferably the formylation reaction is conducted at an increased gas pressure, such as about 20 to about 100 bar, more preferred about 40 to about 60 bar, e.g. at about 50 bar.

The formylation reaction is preferably carried out until completion of the reaction, e.g. for about 48 to about 72 hours.

How to obtain the compounds of the formula VIII is known in the art, e.g. from WO 2006/067456, and is also further illustrated in the examples of the present invention.

The compound of formula I prepared by the process of the present invention is intended to be subjected to a Wittig reaction to obtain substituted pyrimidine derivatives as described above, in particular for the preparation of rosuvastatin.

Preferably, the process of the present invention therefore further comprises the step of reacting the compound of the formula V with a compound of the formula IV

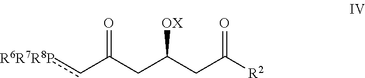

or a salt thereof, wherein R² is OH, OR³, wherein R³ is a carboxyl protecting group, or NR⁴R⁵, wherein R⁴ and R⁵ are independently H or an amido protecting group, X is H or a hydroxy protecting group and R⁶, R⁷ and R⁸ are chosen such that the compound of the formula IV is a Wittig reagent or a Horner-Wittig reagent,
to obtain a compound of the formula VI

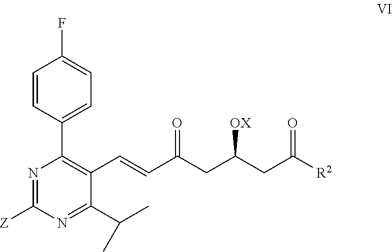

or a salt thereof, wherein R², X and Z are defined as above.

Residue R² within the compounds of the present invention is independently selected from OH, OR³ and NR⁴R⁵, wherein R³ is a carboxyl protecting group and R⁴ and R⁵ are independently H or an amido protecting group.

As protecting groups for the optionally protected hydroxy groups, the optionally protected carboxyl groups and the optionally protected amido groups usual protecting groups known to the person skilled in the art may be used. Suitable protecting groups are exemplified in WO 03/044011, the content of which is incorporated herein by reference.

Preferred protecting groups for X, X′, R³, R⁴ and R⁵ are alkyl, aryl and aralkyl, such as straight, branched or cyclic C₁₋₁₀ alkyl, preferably C₁₋₆ alkyl, more preferably methyl, ethyl, isopropyl, or tert-butyl. Aryl can be for example phenyl or naphthyl. Aralkyl can be for example aryl such as phenyl or naphthyl linked via a C₁₋₁₀ alkyl, preferably C₁₋₆ alkylene, such as benzyl. More preferred X and/or X′ is a tri(C₁₋₆ alkyl)silyl or a diarylalkylsilyl, even more preferred a trimethylsilyl, a tert-butyldimethylsilyl or a diphenyl(tert-butyl)silyl group.

In one preferred embodiment of the present invention R² is OR³ and R³ is alkyl, aryl or aralkyl, preferably R³ is a C₁₋₆ alkyl group, most preferred R³ is a methyl, ethyl or tert-butyl group or R² is NR⁴R⁵ and R⁴ and R⁵ are independently H, alkyl, aryl or an aralkyl group, preferably R⁴ is a C₁₋₆ alkyl group and R⁵ is H, most preferred R⁴ is a tert-butyl group and R⁵ is H, and X is H or a hydroxy protecting group, in particular X is H or a SiPh₂t-Bu group, whereby “Ph” means a phenyl group.

How to obtain the compound of the formula IV is known in the art.

How to choose the residues R⁶, R⁷ and R⁸ so that the compound of the formula IV is a Wittig reagent or a Horner-Wittig reagent or derivatives thereof is known to the person skilled in the art. Suitable selections of residues R⁶, R⁷ and R⁸ are exemplified in German patent application No. 10 2005 022 284.6, the content of which is incorporated herein by reference. In particular in a usual Wittig reagent R⁶, R⁷ and R⁸ are phenyl residues and the bond of the phosphorus atom to the carbon chain is a double bond, and in a usual Horner-Wittig reagent R⁶ and R⁷ are both ethoxy residues and R⁸ is an oxygen, bound to the phosphorus atom by a double bond, i.e. R⁸ is a O=residue, and the phosphorus atom is bound to the carbon chain by a single bond. A Horner-Wittig reagent means a reagent to conduct a Horner-Wadsworth-Emmons-reaction, which is known in the art.

The reaction of the compound of the formula V with a compound of the formula IV, i.e. the Wittig reaction or the Horner-Wittig reaction can be conducted in solvents and under conditions as usually applied and known in the art. As suitable solvents each solvent used to conduct the Wittig reaction can be used, preferably an apolar and aprotic solvent, such as MeCN or toluene, which are preferred. The reaction is typically conducted until completion, e.g. for 4 to 48 hours.

The process of the present invention can be furthermore supplemented by hydrogenating and optionally deprotecting and/or protecting any protected or unprotected group of a compound of the formula VI, obtained by the above described process, in order to obtain a compound of the formula VII

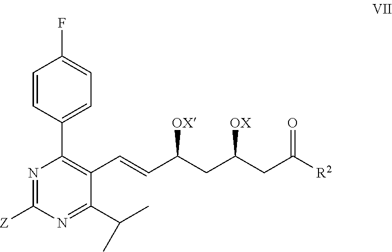

or a salt thereof, wherein X′ is H or a hydroxy protecting group and X, R² and Z are defined as above.

How to conduct the hydrogenation reaction and the optional deprotecting and/or protecting reactions to obtain a compound of the formula VII by reacting a compound of the formula VI is known to the person skilled in the art. It is also known how to deprotect and/or protect any protected or unprotected group of the concerned compounds. Typically silicium containing protecting groups are removed by using an aqueous solution of HF, e.g. using MeCN as solvent. The hydrogenating reaction is typically conducted using a compound of the formula VI, wherein X is hydrogen by reacting such compound with a boron-containing reducing agent, e.g. Et₂BOMe and NaBH₄ in a suitable solvent. Further suitable reducing agents, in particular such to obtain the stereo chemistry of the compound of the formula VII as indicated, and the suitable reaction conditions are known in the art.

Preferably, the compound of the formula VII is modified such that X′ and X are both hydrogen, R² is OH and Z is a —NMeSO₂Me group, such that the compound of the formula VII is rosuvastatin.

In one preferred embodiment in the process of the present invention Z is —NMeSO₂Me or Z is converted into a —NMeSO₂Me group prior to reaction of the compound of the formula IV with a compound of the formula V, and is most preferably such process that in the compound of the formula VI Z is also a —NMeSO₂Me group.

In one embodiment the process of the present invention further comprises a step of converting the residue Z in any of the compounds VI or VII into a —NMeSO₂Me group, if residue Z is different to a —NMeSO₂Me group.

The present invention further relates to a compound of the formula IX

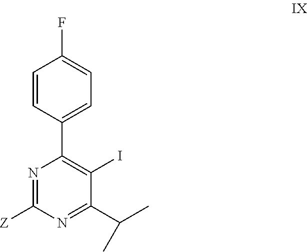

wherein Z is defined as above, which compound is present in a crystalline form. Preferably residue Z is a —NMeSO₂Me group within the compound of the formula IX, which is present in a crystalline form.

The compound of the formula IX, which is present in a crystalline form, can be advantageously used in the process of the present invention, in particular as the compound of the formula IX can be excellently purified by crystallization or by column chromatography (see procedure below) and the use of the compound of the formula IX in a crystalline form in the process of the present invention therefore leads to increased yields.

The present invention also relates to the use of a compound of the formula IX, which is present in crystalline form, for the preparation of rosuvastatin.

Within this application, all starting materials, intermediates and products to be used in the processes of the present invention may be used as racemates or enantiomerically enriched mixtures, e.g. mixtures which are enriched in one enantiomer or comprise only one substantially purified enantiomer.

Each process of the present invention can further comprise one or more steps of separation or enrichment of enantiomers, e.g. steps of racemic separation. Methods of separation or enrichment of enantiomers are known in the art.

Preferably, the stereo configuration of starting materials, intermediates and products is chosen such that when used in processes of the present invention the intermediates and products resulting from said processes show the stereo configuration suitable for the preparation of rosuvastatin or are in or correspond to the stereo configuration of rosuvastatin.

The present invention will now be further illustrated by the following examples which are not intended to be limiting.

Within the examples, reactions and manipulations involving air and moisture-sensitive compounds were performed under an atmosphere of dry argon, using standard Schlenk techniques. Commercial reagents were used without additional purification. Solvents were distilled from appropriate drying agents before use. Chromatographic purification of the products was accomplished using flash column chromatography on Macherey-Nagel silica gel 60 (230-400 mesh ASTM) and on neutral Al₂O₃ with various mixtures of solvents as mobile phases. Thin layer chromatography (TLC) was carried out on Merck plates with aluminium backing and silica gel 60 F₂₅₄. NMR spectra were recorded with Bruker ARX 400 and/or Bruker ARX 300 spectrometers. Chemical shifts are reported in ppm (δ) and referred to internal TMS for ¹H NMR, deuterated solvents for ¹³C NMR. Elemental and mass spectrometric analyses were performed according to standard techniques.

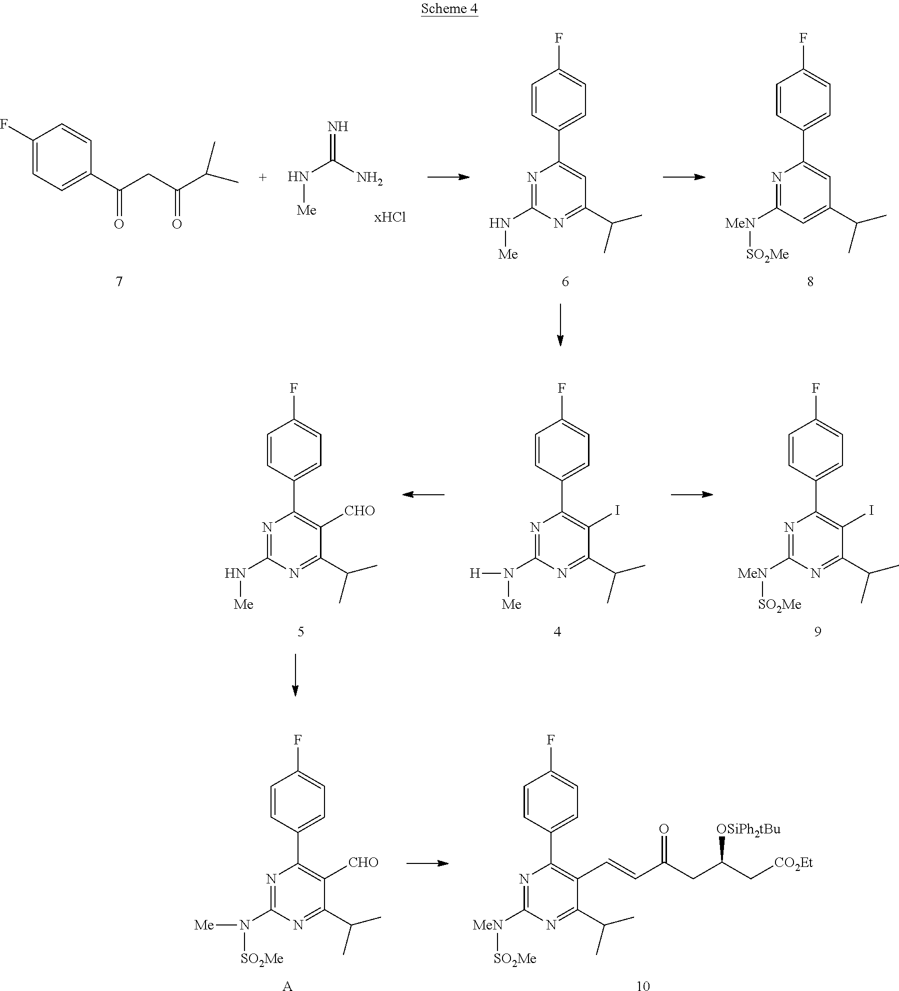

Scheme 4 indicates the reactions as described within the examples.

### EXAMPLE 1

### 4-(4-Fluorophenyl)-6-isopropyl-N-methylpyrimidin-2-amine (6)

Metallic Na (0.21 g, 9 mmol) was added to 35 ml of anhydrous iPrOH (isopropanol) and the suspension was heated at 80° C. until all the metal dissolved. The solution was cooled to 70° C. and 1-methylguanidine hydrochloride (1 g, 9 mmol) was added. The suspension was heated at 82° C. during 2.5 h and then cooled again to 70° C. The solution of 1,3-diketone 7 (1.9 g, 9 mmol) (prepared according to T. Ruman et al., Eur. J. Inorg. Chem., 2003, 13, 2475-2485) in 10 ml of iPrOH then was added and the resulting mixture was heated at 82° C. for 11 h. After cooling to RT the reaction mixture was concentrated in vacuum and residue was diluted with 20 ml of saturated aqueous NH₄Cl. The crude product was extracted with EtOAc (3×10 ml) and combined organic phase was dried over MgSO₄ and concentrated. The residue was purified on a silica-gel column by using n-hexane/EtOAc=9:1, Rf=0.16 or n-hexane/EtOAc=1:1, Rf=0.45 to afford the product 6 (1.44 g, 65%) as colorless powder. M.p. 75-76° C. ¹H-NMR (400 MHz, CDCl₃): δ=1.30 (d, 6H, J=6.8 Hz, Me₂CH), 2.86 (m, 1H, Me₂CH), 3.08 (d, 3H, J=5.1 Hz, MeNH), 5.15 (bs, 1H, NH), 6.82 (s, 1H, CH, pyrimidine), 7.14 (m, 2H, CH, Ar), 8.05 (m, 2H, CH, Ar). ¹³C-NMR (100 MHz, CDCl₃): δ=21.75 (Me₂CH), 28.41 (MeNH), 36.16 (Me₂CH), 102.92 (CH, pyrimidine), 115.50 (d, J_{CF}=21.14 Hz, CH), 128.89 (CH), 128.98 (CH), 134.26 (C), 163.23 (CH), 163.63 (CH), 164.12 (d, J_{CF}=249.96 Hz, CF), 177.26 (C—CHMe₂). MS (El, C₁₄H₁₆FN₃, M=245.3 g/mol), m/z=245 (M⁺, 76), 230 (100), 217 (83), 201 (14), 173 (11), 146 (12); anal. calcd. for C₁₄H₁₆FN₃: C, 68.55; H, 6.57; N, 17.13. found: C, 69.10; H, 6.30; N, 16.57.

### EXAMPLE 2

### N-(4-(4-Fluorophenyl)-6-isopropylpyrimidin-2-yl)-N-methylmethane-sulfonamide (8)

To a solution of amine 6 (0.6 g, 2.45 mmol) and Et₃N (0.32 g, 3.2 mmol) in 20 ml of dry CH₂Cl₂ at 0° C. a solution of MeSO₂Cl (0.28 g, 2.45 mmol) in 5 ml of dry CH₂Cl₂ was added. Reaction mixture was warmed to RT and stirred additionally 5 h. Solvent was evaporated and residue was dried in high vacuum. The purification by column chromatography (silica, toluene/EtOAc=10:1, Rf=0.45 or n-hexane/EtOAc=4:1, Rf=0.22) afforded 8 (237 mg, 30%) as a colorless solid. M.p. 138-139° C. ¹H-NMR (300 MHz, CDCl₃): δ=1.33 (d, 6H, J=6.8 Hz, Me₂CH), 3.02 (m, 1H, Me₂CH), 3.55 (s, 3H, Me-N), 3.62 (s, 3H, MeSO₂—N), 7.18 (m, 3H, CH, Ar+pyrimidine), 8.08 (m, 2H, CH, Ar). ¹³C-NMR (75 MHz, CDCl₃): δ=21.77 (Me₂CH), 28.25 (Me-N), 36.22 (Me₂CH), 42.29 (MeSO₂—N), 107.86 (CH, pyrimidine), 115.67 (d, J_{CF}=23.23 Hz), 129.13 (CH), 129.25 (CH), 129.36 (C), 163.23 (CH), 159.25 (CH), 163.66 (d, CF, J_{CF}=249.96 Hz), 177.49 (Me₂CH—C). MS (El, C₁₅H₁₈FN₃O₂S, M=323.4 g/mol) m/z=323 (M⁺, 8), 308 (12), 245 (41), 244 (100), 230 (47), 217 (30), 57 (19).

### EXAMPLE 3

### 4-(4-Fluorophenyl)-5-iodo-6-isopropyl-N-methylpyrimidin-2-amine (4)

A solution of 6 (1.08 g, 4.4 mmol) and elemental iodide (I₂) (2.24 g, 8.8 mmol) in 25 ml of DMSO was heated at 100° C. during 3 hours and was left overnight at RT. Reaction mixture was diluted with 25 ml of water, extracted with EtOAc (3×15 ml). Combined extracts were washed successively with 1N solution of Na₂S₂O₃ (2×10 ml), saturated NaHCO₃ (2×10 ml), brine (2×10 ml) and then dried over MgSO₄. After evaporation of the solvent, the mixture of crude product and unreacted initial compound was separated by column chromatography (silica, toluene/EtOAc=10:1, Rf=0.54 and/or silica, n-hexane/EtOAc=1:1, Rf=0.61). Additional recrystallization from CHCl₃ afforded pure 4 (0.461 g, 33%) as yellowish crystals. M.p. 195-196° C. ¹H-NMR (400 MHz, CDCl₃): δ=1.26 (d, 6H, J=6.69, Hz Me₂CH), 2.99 (d, 3H, J=4.95 Hz, MeNH) 3.47 (m, 1H, Me₂CH), 5.15 (bs, 1H, NH), 7.12 (m, 2H, CH, Ar), 7.52 (m, 2H, CH, Ar). ¹³C-NMR (100 MHz, CDCl₃): δ=21.12 (Me₂CH), 28.41 (Me-NH), 38.26 (Me₂CH), 80.91 (C—I, pyrimidine), 114.89 (d, J_{CF}=21.93 Hz, CH, Ar), 130.84 (CH), 130.92 (CH), 138.13 (C), 162.04 (CH), 168.77 (CH), 163.00 (d, C—F, J_{CF}=248.72 Hz), 177.24 (Me₂CH—C). MS (El, C₁₄H₁₅FIN₃, M=371.19 g/mol), m/z=371 (M⁺, 87), 356 (20), 245 (22), 244 (100), 146 (14).

### EXAMPLE 4

### N-(4-(4-Fluorophenyl)-5-iodo-6-isopropylpyrimidin-2-yl)-N-methylmethane-sulfonamide (9) (compound of formula VIII)

A solution of amine 4 (20 mg, 0.054 mmol) and Et₃N (7.1 mg, 0.07 mmol) in 2 ml of dry CH₂Cl₂ was cooled to 0° C. and the solution of MeSO₂Cl (6.2 mg, 0.054 mmol) in 0.5 ml of dry CH₂Cl₂ was added. The reaction mixture was warmed up to RT and stirred for 1.5 h. Solvent was evaporated and crude product was purified by column chromatography (silica, toluene/EtOAc=10:1, Rf=0.40 or n-hexane/EtOAc=4:1, Rf=0.31). Yield of 9 (7.3 mg 30%). ¹H-NMR (300 MHz, CDCl₃): δ=1.30 (d, 6H, J=6.59 Hz, Me₂CH), 3.02 (m, 1H, Me₂CH), 3.39 (s, 3H, Me-N), 3.47 (s, 3H, MeSO₂N), 7.16 (m, 2H, CH, Ar+pyrimidine), 8.10 (m, 2H, CH, Ar).

### EXAMPLE 5

### 4-(4-Fluorophenyl)-6-isopropyl-2-(methylamino)pyrimidine-5-carbaldehyde (5) (compound of formula V)

Under argon a 10 ml flask was charged with Pd(OAc)₂ (29.6 mg, 0.132 mmol), nBuAd₂P (14.3 mg, 0.04 mmol) and 4 ml of toluene. Resulted mixture was vigorously stirred for 1-1.5 h at RT and N,N,N′,N′-tetramethylethylendiamine (TMEDA) (34.9 mg, 0.3 mmol) and iodide 4 (148.5 mg, 0.4 mmol) were added. Resulting solution was placed into 25 ml autoclave, equipped with a magnetic stirring bar. The autoclave was flushed 3 times with mixture CO/H₂ (1:1) and pressurized with CO/H₂ (1:1) to 50 bar. The reaction mixture was stirred at 100° C. for 72 h. After cooling to RT and releasing of the excess CO/H₂, the solvent was evaporated and the crude product was purified by column chromatography (silica, toluene/EtOAc=10:1, Rf=0.44) to give 5 (76.5 mg, 70%) as colorless solid. M.p. 131-132° C. ¹H-NMR (400 MHz, CDCl₃): δ=1.26 (d, 6H, J=6.60 Hz, Me₂CH), 3.11 (d, 3H, J=5.11 Hz, MeNH) 4.03 (m, 1H, Me₂CH), 5.62 (bs, 1H, NH), 7.17 (m, 2H, CH, Ar), 7.54 (m, 2H, CH, Ar), 9.82 (s, 1H, CHO). ¹³C-NMR (100 MHz, CDCl₃): δ=21.36 (Me₂CH), 28.31 (Me-NH), 38.26 (Me₂CH), 115.58 (d, J_{CF}=23.90 Hz, CH, Ar), 130.35 (CH), 131.67 (CH), 137.86 (C), 162.05 (CH), 168.41 (CH), 163.00 (d, CF, J_{CF}=246.5 Hz), 177.36 (Me₂CH—C), 190.12 (CHO). MS (El, C₁₅H₁₆FN₃O, M=273.31 g/mol) m/z=273 (100), 256 (33), 244 (20), 230 (63), 217 (77); anal. calcd. for C₁₅H₁₆FN₃O: C, 65.92; H, 5.90. found: C, 65.80; H, 6.34.

### EXAMPLE 6

### N-(4-(4-Fluorophenyl)-5-formyl-6-isopropylpyrimidin-2-yl)-N-methylmethanesulfonamide (A)

Method 1 To solution of aldehyde 5 (40 mg, 0.15 mmol) and Et₃N (24.9 mg, 0.25 mmol) in 3 ml of dry CH₂Cl₂ at 0° C. was added a solution of MeSO₂Cl (18.9 mg, 0.16 mmol) in 1 ml of dry CH₂Cl₂. Reaction mixture was warmed to RT and stirred additionally 2 h at this temperature. Solvent was evaporated and residue was purified by column chromatography (silica, toluene/EtOAc=10:1, Rf=0.52) to afford aldehyde A (15.5 mg 30%) as colorless solid.

Method 2 To solution of aldehyde 5 (60 mg, 0.22 mmol) in 1 ml dry DMF at 0° C. sodium hydride (NaH) (11 mg, 0.46 mmol) was added. The solution was stirred for 30 min and then solution of MeSO₂Cl (37.7 mg, 0.336 mmol) in 1 ml of DMF was added. Resulting mixture was stirred for 30 min at 0° C. and 3 h at RT. Then 2 ml of water was added to quench the reaction mixture and extracted with EtOAc (3×3 ml). The organic layer was washed with brine, dried over MgSO₄. After evaporation of the solvent, the product was purified by column chromatography (silica, toluene/EtOAc=10:1, Rf=0.52) to give 42.4 mg (55%) of aldehyde A as colorless solid. M.p. 147-148° C. ¹H-NMR (300 MHz, CDCl₃): δ=1.32 (d, 6H, Me₂CH, J=6.62 Hz), 3.55 (s, 3H, MeN), 3.64 (s, 3H, MeSO₂—N), 4.03 (m, 1H, Me₂CH), 7.23 (m, 2H, CH, Ar), 7.63 (m, 2H, CH, Ar), 9.97 (bs, 1H, CHO). ¹³C-NMR (100 MHz, CDCl₃): δ=21.38 (Me₂CH), 28.32 (MeN), 38.26 (Me₂CH), 42.29 (MeSO₂N), 115.58 (d, J_{CF}=23.5 Hz, CH, Ar), 130.35 (CH), 131.7 (CH), 137.86 (C), 162.05 (CH), 168.41 (CH), 163.00 (d, CF, J_{CF}=246.5 Hz), 177.36 (Me₂CH—C), 190.12 (CHO). MS (El, C₁₆H₁₈FN₃O₃S, M=351.4 g/mol) m/z=351 (22), 273 (18), 272 (100).

### EXAMPLE 7

### Ethyl (R)-3-(tert-butyldiphenylsilyloxy)-7-(4-(4-fluorophenyl)-6-isopropyl-2-(N-methylmethylsulfonamido)pyrimidin-5-yl)-5-oxohept-6-(E)-enoate (10)

Method 1 (according to modified procedure given in M. Watanabe et al., Bioorg. Med. Chem. 1997 5(2), 437-444.) Solution of aldehyde A (16 mg, 0.046 mmol) and ylide (R)-B (wherein X=tert-butyldiphenylsilyloxy) (30 mg, 0.046 mmol) (obtainable by methods known in the art, e.g. as described in U.S. Pat. No. 5,620,440) in 1 ml of MeCN was reflux for 14 h. Solvent was removed in vacuum and crude product was purified by column chromatography (silica, EtOAc, Rf=0.92) yielding 10 (24 mg, 70%) as viscous oil.

Method 2 Solution of aldehyde A (16 mg, 0.046 mmol) and ylide (R)-B (same as with method 1) (30 mg, 0.046 mmol) in 1 ml of toluene was reflux during 48 h and evaporated under residue pressure to remove toluene. Product was purified by column chromatography (silica, EtOAc, Rf=0.92) affording 10 18 mg (52%) as colorless viscous oil. ¹H-NMR (300 MHz, CDCl₃): δ=1.00 (s, 9H, Me₃C), 1.26 (m, 3H+6H, Me₂CH+CH₂Me), 3.52 (s, 3H, Me-N), 3.59 (s, 3H, MeSO₂N), 2.49 (m, 2H, CH₂CO₂Et), 2.69 (dd, 1H, J=15.75 Hz, J=5.53 Hz, C(O)CHH), 2.83 (dd, 1H, J=15.75 Hz, J=7.48 Hz, C(O)CHH), 3.25 (m, 1H, Me₂CH), 4.05 (q, J=7.11 Hz, CH₂Me), 4.59 (m, 1H, CH), 5.95 (d, 1H, J=16.50 Hz, CH═CH), 7.07 (m, 2H, Ar), 7.36 (m, 7H, CH, CH═CH+Ar), 7.55 (m, 2H, CH, Ar), 7.65 (m, 4H, CH, Ar).

1. A process for the preparation of a compound of the formula V 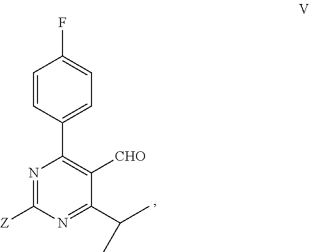
wherein Z is an —NMeSO₂Me group or a group capable of being converted into an —NMeSO₂Me group,
wherein said process comprises the steps of
a) formylating a compound of the formula VIII 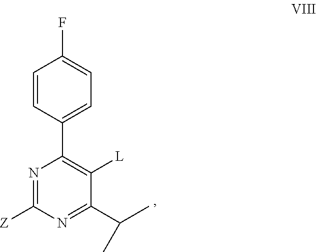
wherein Z is defined as above and L is a leaving group and
b) optionally converting Z into a —NMeSO₂Me group.

2. The process according to claim 1, wherein the formylation step is carried out in the presence of a catalyst.

3. The process according to claim 2, wherein the catalyst is a palladium based catalyst.

4. The process according to claim 2, wherein the formylation reaction is conducted using H₂ and CO.

5. The process according to claim 1, wherein L is iodine.

6. The process according to claim 1, further comprising the step of reacting the compound of the formula V with the compound of the formula IV 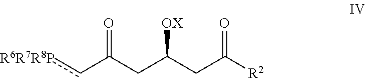
or a salt thereof, wherein R² is OH, OR³, wherein R³ is a carboxyl protecting group, or NR⁴R⁵, wherein R⁴ and R⁵ are independently H or an amido protecting group, X is H or a hydroxy protecting group and R⁶, R⁷ and R⁸ are chosen such that the compound of the formula IV is a Wittig reagent or a Horner-Wittig reagent,
to obtain a compound of the formula VI 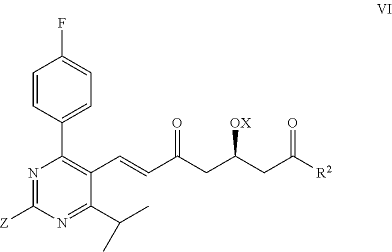
or a salt thereof, wherein R² and X are defined as above and Z is defined as in claim 1.

7. The process according to claim 6, further comprising the step of hydrogenating and optionally deprotecting and/or protecting a protected or unprotected group of a compound of formula VI to obtain a compound of the formula VII 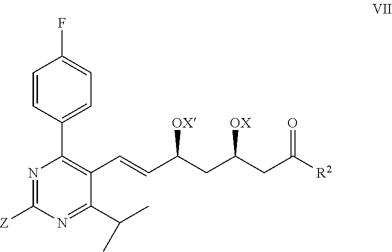
or a salt thereof, wherein X′ is H or a hydroxy protecting group and X, R² and Z are defined as in claim 6.

8. The process according to claim 1, further comprising the step of converting Z into a —NMeSO₂Me group.

9. The compound of the formula IX 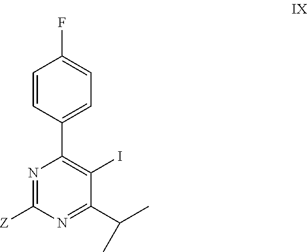
wherein Z is an —NMeSO₂Me group or a group capable of being converted into an —NMeSO₂Me group, wherein said compound is present in a crystalline form.

10. A method of preparing rosuvastatin using the compound of the formula IX as defined in claim 9.
